(19) United States
(12) Patent Application Publication
(10) Pub. No.: US 2003/0114536 A1
(43) Pub. Date: Jun. 19, 2003

(54) Venlafaxine hydrochloride monohydrate and methods for the preparation thereof

(75) Inventors: Jun Han, Park City, IL (US); Yong Jai Lee, Monroe, NY (US)

Correspondence Address: Rebecca R. Barrett, 5 Giralda Farms, Madison, NJ, 07940, US

(73) Assignee: Wyeth, Madison, NJ

(21) Appl. No.: 10/308,472

(22) Filed: Dec. 3, 2002

Related U.S. Application Data
(60) Provisional application No. 60/335,823, filed on Dec. 5, 2001.

(51) Int. Cl. A61K 31/137; C07C 215/28

(52) U.S. Cl. 514/649; 564/316

(57) ABSTRACT

This invention relates to novel crystalline polymorphic form of venlafaxine hydrochloride which exists in hydrated form (e.g., as a monohydrate), methods for the preparation thereof, and its use.

This application claims priority from copending provisional application serial No. 60/335,823, filed on Dec. 5, 2001, the entire disclosure of which is hereby incorporated by reference.

### FIELD OF THE INVENTION

This invention relates to a novel crystalline polymorphic form of venlafaxine hydrochloride which exists in hydrated form (e.g., as a monohydrate), methods for the preparation thereof, and its use.

### BACKGROUND OF THE INVENTION

Venlafaxine (1-[2-(dimethylamino)-1-(4-methoxyphenyl)ethyl]cyclohexanol) and its therapeutically acceptable salts (collectively referred to as venlafaxine herein) are inhibitors of monoamine neurotransmitter uptake, a mechanism associated with clinical antidepressant activity. This mechanism has also been associated with reproductive function by affecting indirectly the hypothalamic-pituitary-ovarian axis. It is believed that venlafaxine's mechanism of action is related to potent inhibition of the uptake of the monoamine neurotransmitters serotonin and norepinephrine. To a lesser degree, venlafaxine also inhibits dopamine reuptake. However, it has no inhibitory activity on monoamine oxidase.

In contrast to classical tricyclic antidepressant drugs, venlafaxine has virtually no affinity for muscaranic, histaminergic, or adrenergic receptors in vitro. Pharmacologic activity at these receptors is associated with the various anticholinergic, sedative, and cardiovascular effects seen with the tricyclic antidepressant drugs.

Hypothalamic amenorrhea in depressed and non-depressed human females may also be treated with venlafaxine as taught in U.S. Pat. No. 5,506,270.

U.S. Pat. No. 5,530,013 teaches that venlafaxine induces cognition enhancement and treats cognitive impairment in a mammal.

U.S. Pat. No. 5,744,474 discloses that venlafaxine can treat urinary incontinence in humans.

More recently, as discussed in U.S. Pat. No. 5,916,923, venlafaxine has been found to treat, prevent, and control obesity, generalized anxiety disorders, post-traumatic stress disorder, late luteal phase disphoric disorder (premenstrual syndrome), attention deficit disorder (with and without hyperactivity), Gilles de la Tourette syndrome, bulimia nervosa, and Shy Drager Syndrome in mammals (e.g., humans).

Extended release formulations of venlafaxine are disclosed in U.S. Pat. No. 6,274,171 and International Patent Publication No. WO 94/27589. As discussed in U.S. Pat. No. 6,274,171, venlafaxine hydrochloride is known to exist in two polymorphic forms, Forms I and II. Characteristic X-ray powder diffraction patterns for Forms I and II are shown in FIGS. 8 and 9, respectively.

### SUMMARY OF THE INVENTION

The present invention provides a novel crystalline polymorph of venlafaxine hydrochloride, which exists in hydrated form (e.g., as a monohydrate) (hereinafter referred to as “the monohydrate form” or “venlafaxine hydrochloride monohydrate”). The monohydrate form is stable in moist environments (e.g., atmospheres having at least 20% relative humidity), such as those often encountered during processing and storage. Furthermore, the monohydrate form is more stable than known forms of venlafaxine hydrochloride in processes involving water, such as wet granulation. Consequently, venlafaxine hydrochloride monohydrate can be subjected to processes involving water without undergoing any solid-state transition.

Venlafaxine hydrochloride monohydrate has characteristic XRPD peaks (expressed in degrees 2θ) at about 7.45, 8.60, 12.93, 14.95, 15.54, 15.84, 16.43, 17.53, 18.02, 18.58, 18.80, 19.54, 20.05, 20.51, 21.41, 22.46, 23.07, 23.52, 24.08, 24.70, 25.44, 26.54, 27.07, 28.29, 30.60, 31.37, 32.25, 32.74, 34.04, and 34.60.

The crystalline polymorph of the present invention can be administered to a mammal to treat depression (including, but not limited to, major depressive disorder, bipolar disorder, and dysthymia), fibromyalgia, anxiety, panic disorder, agoraphobia, post traumatic stress disorder, premenstrual dysphoric disorder (premenstrual syndrome), attention deficit disorder (with and without hyperactivity), obsessive compulsive disorder (including trichotillomania), social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction (including premature ejaculation), borderline personality disorder, chronic fatigue syndrome, urinary incontinence, pain (including, but not limited to, migraine, chronic back pain, phantom limb pain, central pain, neuropathic pain such as diabetic neuropathy, and postherpetic neuropathy), Shy Drager Syndrome, or Raynaud's syndrome. The crystalline polymorph can also be administered to prevent relapse or recurrence of depression, to induce cognitive enhancement, to treat cognitive impairment, and in regimens for cessation of smoking or other tobacco uses. Additionally, the crystalline polymorphs can be administered to treat hypothalamic amenorrhea in depressed and non-depressed human females. These methods involve administering to a mammal (e.g., a human) in need thereof an effective amount of the crystalline polymorph of the present invention or a mixture of venlafaxine polymorphs that contains the crystalline polymorph of the present invention. Preferably, the venlafaxine is administered orally.

Another embodiment is a pharmaceutical composition comprising the crystalline polymorph of the present invention and, optionally, a pharmaceutically acceptable carrier or diluent. Typically, the pharmaceutical composition comprises an amount of the crystalline polymorph effective to treat depression or any of the aforementioned indications in an animal, such as a mammal (e.g. human). According to one preferred embodiment, the pharmaceutical composition comprises at least about 20, 30, 40, 50, 60, 70, 80, 90, 95, 96, 97, 98, 99, 99.1, 99.2, 99.3, 99.4, 99.5, 99.6, 99.7, 99.8, or 99.9% by weight of the monohydrate form of the crystalline polymorph of the present invention, based upon 100% total weight of venlafaxine hydrochloride in the pharmaceutical composition.

The pharmaceutical composition may be incorporated into a dosage form, such as a tablet or capsule.

The monohydrate form can be prepared by wet granulating Form I or II of venlafaxine hydrochloride or a mixture thereof. Also, the monohydrate form can be prepared by recrystallizing Form I or II of venlafaxine hydrochloride or a mixture thereof from water or a mixture of water and an organic solvent.

In each of the aforementioned methods of preparing the crystalline polymorph of the present invention, the crystals formed may be recovered by any method known in the art.

### BRIEF DESCRIPTION OF THE DRAWINGS

FIG. 1 is a characteristic X-ray Powder Diffraction (XRPD) pattern of venlafaxine hydrochloride monohydrate.

FIG. 2 is a differential scanning calorimetry (DSC) scan of venlafaxine hydrochloride monohydrate carried out in a sealed pan at a scan rate of 10° C./minute from 25° C. to 240° C. under a nitrogen purge.

FIG. 3 is a DSC scan of venlafaxine hydrochloride monohydrate carried out in a vented pan at a scan rate of 10° C./minute from 25° C. to 240° C. under a nitrogen purge.

FIG. 4 is a thermogravimetric analysis (TGA) of venlafaxine hydrochloride monohydrate heated from 25 to 230° C. at a scan rate of 10° C./minute under a nitrogen purge.

FIG. 5 is a graph of two water sorption cycles of venlafaxine hydrochloride monohydrate carried out at 25° C.

FIG. 6 are XRPD patterns of the novel crystalline polymorph form of venlafaxine hydrochloride of the present invention in its monohydrate form and after being dehydrated and rehydrated.

FIG. 7 are graphs of the intrinsic dissolution of venlafaxine hydrochloride Form II and venlafaxine hydrochloride monohydrate over time.

FIG. 8 is a characteristic XRPD pattern of Form I of venlafaxine hydrochloride.

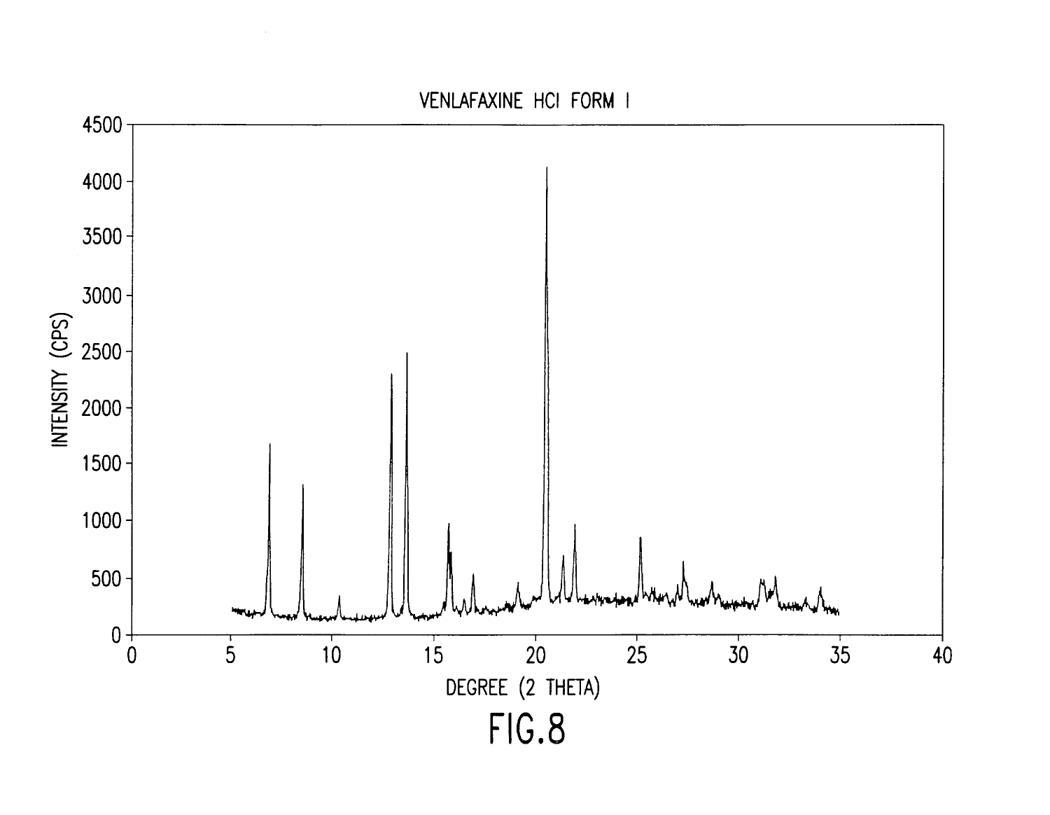

FIG. 9 is a characteristic XRPD pattern of Form II of venlafaxine hydrochloride.

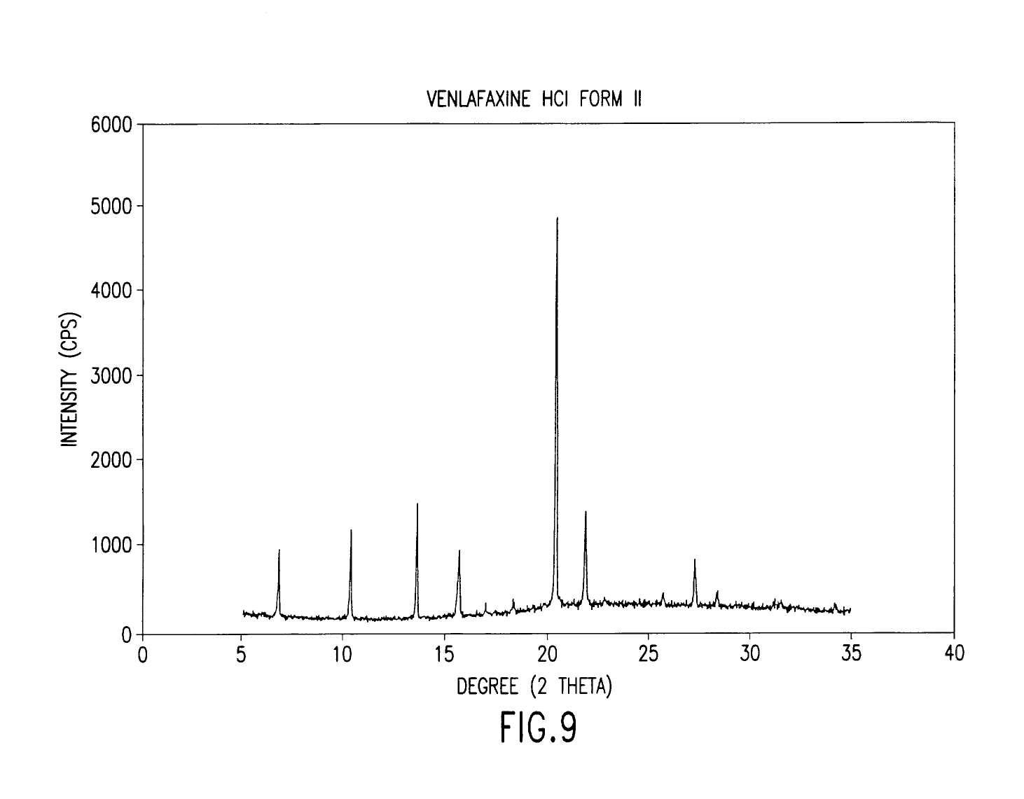

### DETAILED DESCRIPTION OF THE INVENTION

The term “about” generally means within 10%, preferably within 5%, and more preferably within 1% of a given value or range. With regard to a given value or range in degrees 2θ from XRPD patterns, the term “about” generally means within 0.2° 2θ and preferably within 0.1°, 0.05°, or 0.01° 2θ of the given value or range. Alternatively, the term “about” means within an acceptable standard error of the mean, when considered by one of ordinary skill in the art.

The term “treat” as used herein refers to preventing, ameliorating, controlling, or curing the desired symptoms or disorders.

The term “venlafaxine hydrochloride” as used herein refers to racemic mixtures of R and S-venlafaxine and their optically pure enantiomers. The crystalline polymorph of the present invention may be R, S, or a racemic mixture of R and S-venlafaxine hydrochloride.

The term “monohydrate” as used herein refers to a hydrate in which one molecule of water is associated with each molecule of venlafaxine hydrochloride.

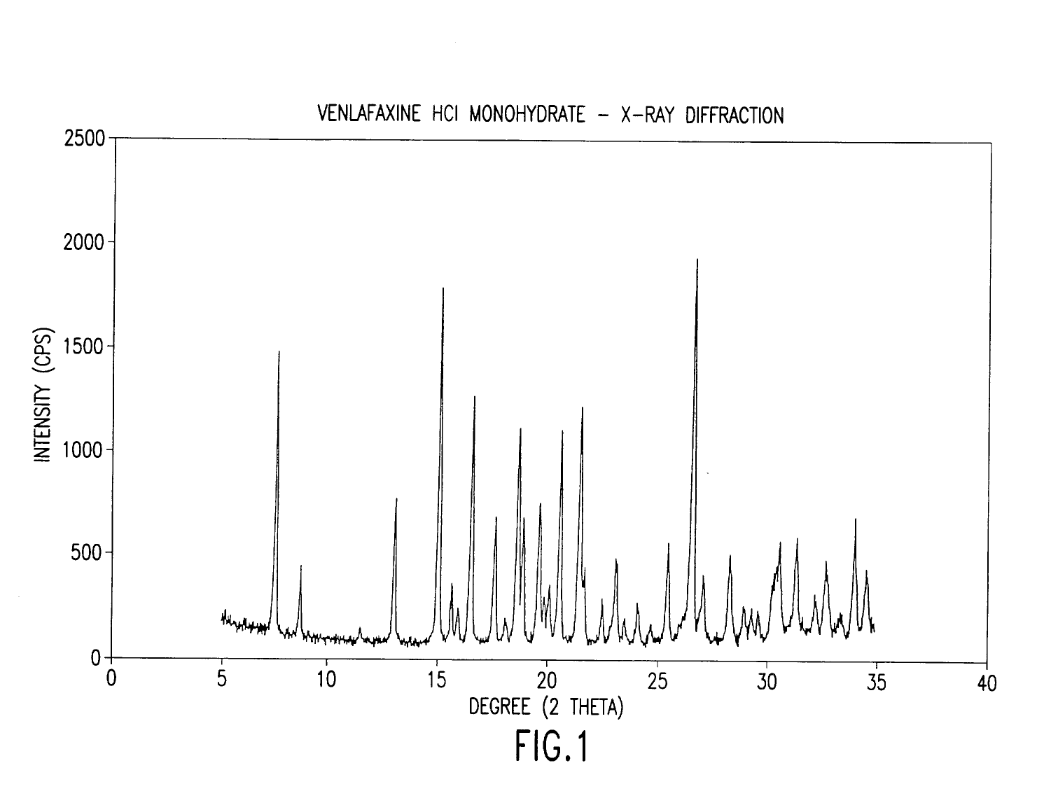

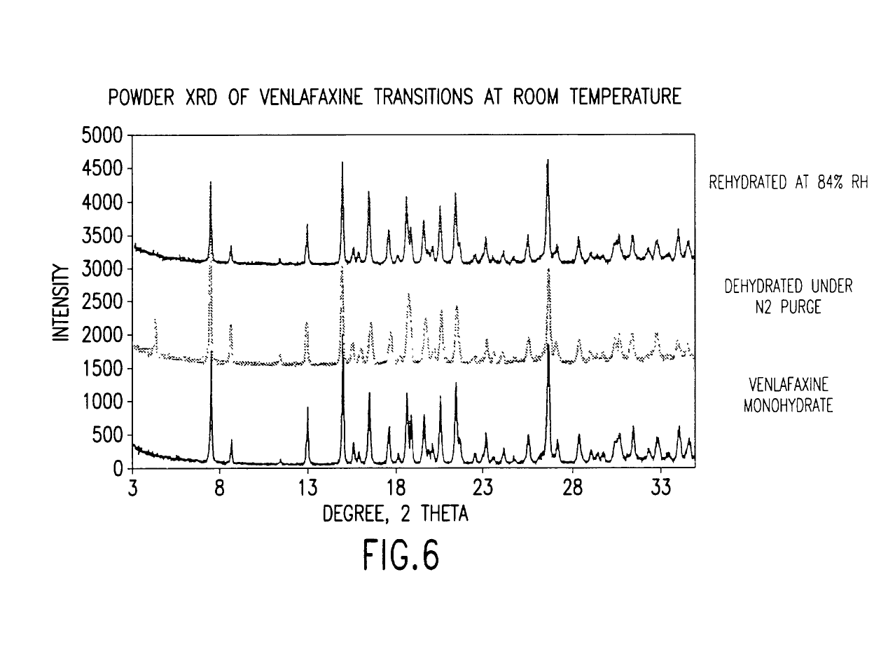

The term “venlafaxine hydrochloride monohydrate” as used herein refers to a monohydrate of venlafaxine hydrochloride (C₁₇H₂₇NO₂.HCl.H₂O) having an XRPD pattern substantially identical to that shown in FIG. 1 or FIG. 6 designated as “Monohydrate”. Peak locations and intensities for the XRPD pattern in FIG. 1 are provided in Table 1 below.

TABLE 1
Characteristic XRPD Peaks (expressed in degrees 2θ) D spacing in
Angstrom (Å) and Intensities of Diffraction Lines in CPS for
Venlafaxine Hydrochloride Monohydrate

| Degrees 2θ | d (Å) | I (Counts per Second (CPS) |
|---|---|---|
| 7.45 | 11.85 | 1468.03 |
| 8.60 | 10.27 | 421.81 |
| 12.93 | 6.84 | 769.96 |
| 14.95 | 5.92 | 1801.74 |
| 15.54 | 5.70 | 336.53 |
| 15.84 | 5.59 | 225.59 |
| 16.43 | 5.39 | 1266.56 |
| 17.53 | 4.92 | 169.81 |
| 18.02 | 4.92 | 169.81 |
| 18.58 | 4.77 | 1107.88 |
| 18.80 | 4.72 | 673.85 |
| 19.54 | 4.54 | 743.85 |
| 20.05 | 4.43 | 333.15 |
| 20.51 | 4.33 | 1112.55 |
| 21.41 | 4.15 | 1215.22 |
| 22.46 | 3.96 | 258.48 |
| 23.07 | 3.85 | 473.17 |
| 23.52 | 3.78 | 174.48 |
| 24.08 | 3.69 | 249.15 |
| 24.70 | 3.60 | 155.81 |
| 25.44 | 3.50 | 552.51 |
| 26.54 | 3.36 | 1896.47 |
| 27.07 | 3.29 | 390.38 |
| 28.29 | 3.15 | 482.86 |
| 30.60 | 2.92 | 542.31 |
| 31.37 | 2.85 | 581.94 |
| 32.25 | 2.77 | 284.69 |
| 32.74 | 2.73 | 463.04 |
| 34.04 | 2.63 | 681.03 |
| 34.60 | 2.59 | 403.59 |

In particular, the peaks (expressed in degrees 20θ) at about 7.45, 8.60, 14.95, 18.02, 21.41, 27.07, 32.74, and 34.60 are unique to venlafaxine hydrochloride monohydrate. Venlafaxine hydrochloride monohydrate has a molecular weight of about 331.88 g/mol. It also has a dehydration endotherm, according to DSC, at about 85° C. and a melting endotherm, according to DSC, at 219-221° C.

Venlafaxine hydrochloride monohydrate generally loses the hydration water at less than 10% relative humidity to form an anhydrous form. The anhydrous form of the crystalline polymorph generally converts to the monohydrate at greater than 10% relative humidity.

The crystalline polymorph of the present invention is useful for treating, preventing, or controlling depression and the aforementioned indications. The appropriate dosage amounts for an animal can be determined by methods known in the art. Generally, a therapeutic effective amount for the desired purpose is administered. The dosage of the crystalline polymorph of venlafaxine hydrochloride disclosed herein is generally from about 75 to about 300 mg per day.

The crystalline polymorph can be formulated into a pharmaceutical composition. Preferably, the pharmaceutical composition comprises an amount of the crystalline polymorph of venlafaxine hydrochloride effective to treat the desired indication in an animal, such as a human. According to one preferred embodiment, the pharmaceutical composition comprises at least about 20, 30, 40, 50, 60, 70, 80, 90, 95, 96, 97, 98, 99, 99.1, 99.2, 99.3, 99.4, 99.5, 99.6, 99.7, 99.8, or 99.9% by weight of venlafaxine hydrochloride monohydrate, based upon 100% total weight of venlafaxine hydrochloride in the pharmaceutical composition.

According to yet another preferred embodiment, the pharmaceutical composition comprises at least about 20, 30, 40, 50, 60, 70, 80, 90, 95, 96, 97, 98, 99, 99.1, 99.2, 99.3, 99.4, 99.5, 99.6, 99.7, 99.8, or 99.9% by weight of venlafaxine hydrochloride monohydrate, based upon 100% total weight of crystalline venlafaxine hydrochloride in the pharmaceutical composition.

The pharmaceutical composition can also be substantially free or completely free of other crystalline polymorphs of venlafaxine hydrochloride, such as Forms I and II. For example, the pharmaceutical composition can contain the monohydrate form in substantially pure form. The terms “substantially free” and “substantially pure” include those pharmaceutical compositions that contain less than 0.01, 0.1, 0.2, 0.3, 0.4, 0.5, 1 or 2% by weight of other crystalline polymorphs, such as Form I or II or both, based upon the total weight of pharmaceutical composition (or alternatively based upon on the total weight of venlafaxine hydrochloride in the pharmaceutical composition).

According to one embodiment, the pharmaceutical composition contains from about 25 to about 350 mg of the crystalline polymorph of venlafaxine hydrochloride. More preferably pharmaceutical compositions of the present invention contain 75 mg, 150 mg or 225 mg of the crystalline polymorth of venlafaxine hydrochloride.

The pharmaceutical composition may also include one or more pharmaceutically acceptable carriers or diluents (e.g. water and organic solvents) and excipients. The term “excipient” includes, but is not limited to, those materials that are acceptable for use in pharmaceutical formulations, and are added to the formulation to promote the stability and viability of the formulation, such as binders, bulking agents, clarifying agents, buffering agents, wetting agents, lubricants, sweeteners, and flavoring agents. Suitable excipients include, but are not limited to, cellulose, ethyl cellulose, gelatin, hydroxypropyl methylcellulose, iron oxide, titanium dioxide, lactose, magnesium stearate, and sodium starch glycolate. Suitable pharmaceutically acceptable carriers, diluents, and excipients also include those described in Remington's, The Science and Practice of Pharmacy, (Gennaro, A. R., ed., 19^{th} edition, 1995, Mack Pub. Co.) which is herein incorporated by reference. The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human).

According to one preferred embodiment, the pharmaceutical composition is an extended release formulation, such as that described in U.S. Pat. No. 6,274,171, which is herein incorporated by reference. For example, an extended release formulation may comprise spheroids comprised of the crystalline polymorph of the present invention, microcrystalline cellulose, and, optionally, hydroxypropylmethylcellulose. The spheroids are preferably coated with a film coating composition comprised of ethyl cellulose and hydroxypropylmethylcellulose.

The pharmaceutical composition may be a dosage form, such as a liquid (e.g., elixirs and suspensions), capsule, pill, or tablet. The pharmaceutical compositions and the crystalline polymorphs of venlafaxine hydrochloride may be administered to animals, including, but not limited to, mammals (e.g. humans), orally, intravenously, intramuscularly, parenterally intraperitoneally, subdermally, buccally, subcutaneously, transdermally, topically, rectally, vaginally, or intranasally. Preferably, the composition is administered orally.

The venlafaxine hydrochloride monohydrate can be prepared by wet granulating Form I or II of venlafaxine hydrochloride or a mixture thereof. It can also be prepared by recrystallizing Form I or II of venlafaxine hydrochloride or a mixture thereof from water or a mixture of water and an organic solvent. A non-limiting example of a suitable organic solvent is ethanol.

Venlafaxine hydrochloride may be prepared by any method known in the art including, but not limited to, the methods described in U.S. Pat. Nos. 4,535,186 and 4,761,501 and International Patent Publication Nos. WO 00/32555, WO 00/32556, and WO 01/07397, all of which are hereby incorporated by reference.

The crystals formed may be recovered by any method known in the art, such as filtration, centrifugation, or with a Buchner style filter, Rosenmund filter, or plates and frame press. Typically, the crystals are recovered as solids.

### EXAMPLES

The following examples are illustrative and are not meant to limit the scope of the claimed invention. The venlafaxine hydrochloride which is used as a raw material in the example below can be prepared by any method known in the art.

### Example 1

### Preparation of Venlafaxine Hydrochloride Monohydrate

Approximately 1.5 mL of water was heated in a beaker to near its boiling point and about 3 g of venlafaxine hydrochloride of Form I or Form II were added. The suspension was stirred with heating until all of the venlafaxine hydrochloride was dissolved. The resulting clear solution was slowly cooled down to ambient temperature. The solution was stored at 5° C. to allow the monohydrate to crystallize. The crystals were removed from the beaker onto filter paper and exposed to the air for about 8 hours to dry. The final product was ground and stored in sealed glass vials.

The product was an odorless, white to off white crystalline powder and had a melting point, as measured by DSC, of ˜219° C.

Differential Scanning Calorimetry (DSC)

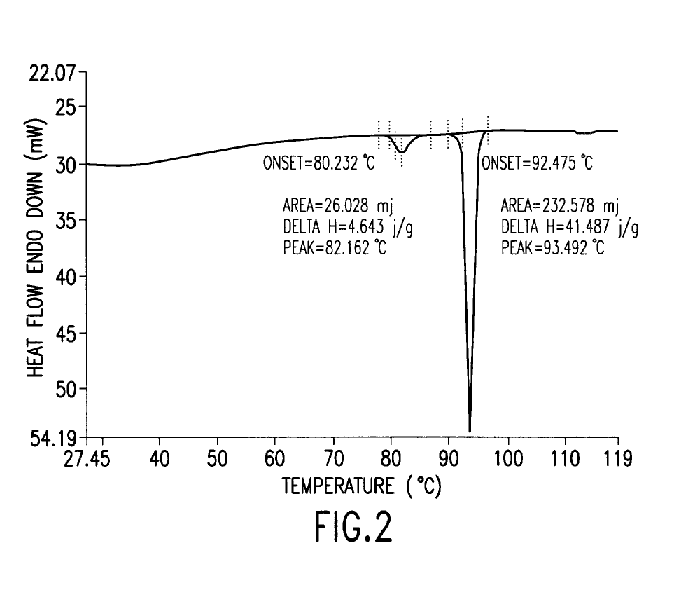

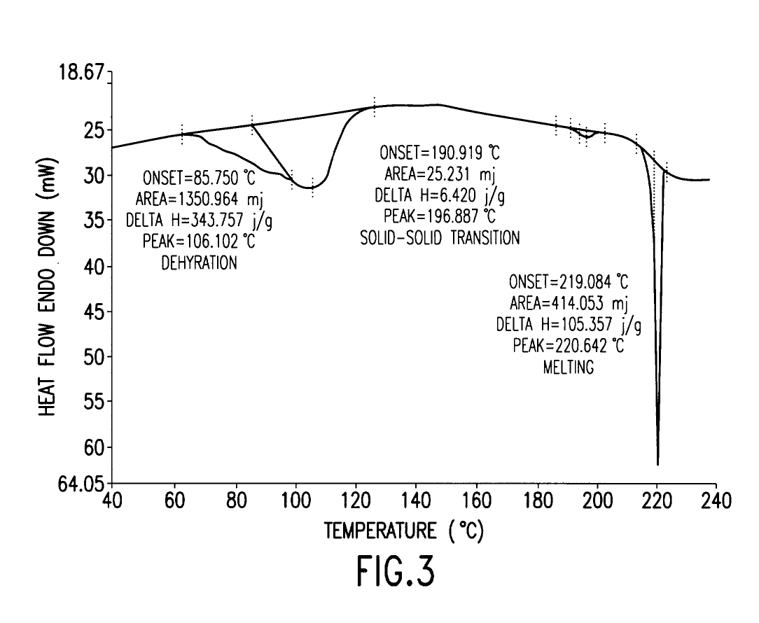

DSC measurements were carried out in both sealed pan and vented pan at a scan rate of 10° C./minute from 25° C. to 240° C. under a nitrogen purge with a Pyris I DSC available from Perkin-Elmer of Shelton, Conn. The DSC scans with the sealed pan and the vented pan are shown in FIGS. 2 and 3, respectively.

FIG. 2 shows a small endotherm at 82° C. and a large endotherm at 93° C. (heat of fusion is 41 J/g), which was the melting of the venlafaxine hydrochloride monohydrate.

FIG. 3 shows three endotherms. The first is a broad endotherm at 106° C., which was due to the dehydration and evaporation of water from the sample. The second is a small endotherm at 196° C. corresponding to a solid-solid phase transition. The third is a large endotherm at 221° C., which was the melting of the anhydrous venlafaxine hydrochloride. The onset melting temperature for venlafaxine hydrochloride was 219° C. (heat of fusion is 105 J/g).

Thermogravimetric Analysis (TGA)

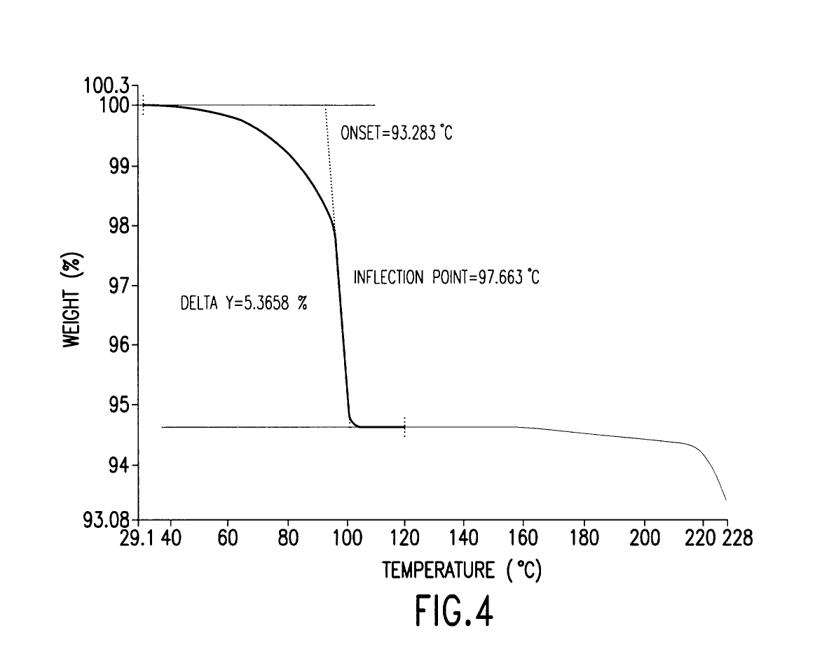

A sample of venlafaxine hydrochloride monohydrate was heated from 25 to 230° C. at a scan rate of 10° C./minute in a Pyris I TGA, available from Perkin-Elmer of Shelton, Conn., under a nitrogen purge. The results are shown in FIG. 4.

At 125° C., the sample lost 5.37% of its weight. The theoretical amount of water in venlafaxine hydrochloride monohydrate is about 5.4% by weight. Therefore, this TGA result agrees with that of a monohydrate of venlafaxine hydrochloride.

Water Vapor Sorption/Desorption

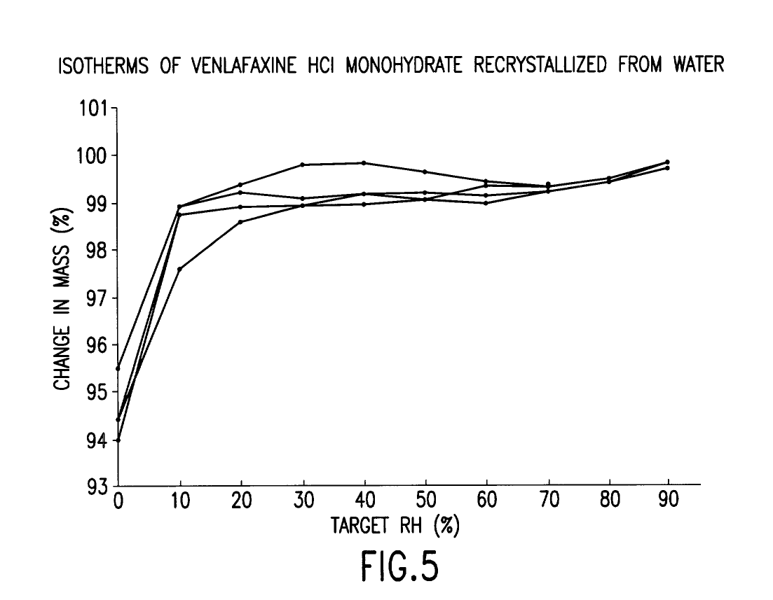

A water vapor sorption/desorption study of venlafaxine hydrochloride monohydrate was carried out in a dynamic vapor sorption instrument, available as DVS-2 from Surface Measurement Systems of London, England. About 30 mg of venlafaxine hydrochloride monohydrate was subjected to a relative humidity-time program at 25° C. The sample weight changes were monitored as function of the time and relative humidity. Two identical relative humidity-time experiment cycles (0 to 90% relative humidity) were performed to detect any changes in solid-state properties. The results are shown in FIG. 5.

As shown in FIG. 5, the venlafaxine hydrochloride monohydrate was stable between 10 and 90% relative humidity and only lost its hydrate water below 10% relative humidity. The compound re-hydrates to the monohydrate form when the relative humidity is above 10%.

On cycling, the compound showed two almost identical water sorption/desorption cycles. This is an indication that the solid-state transition of venlafaxine hydrochloride monohydrate was reversible during the dehydration/hydration processes, i.e., there was no recrystallization of the anhydrate/hydrate phase.

X-ray Powder Diffraction (XRPD)

XRPD was performed on the venlafaxine hydrochloride monohydrate under both ambient and dry conditions with a Scintag X2 X-Ray Diffraction System Model 00-A02, available from Thermo ARL of Ecublens, Switzerland. The XRPD instrument had the following parameters:

| Scan Type: | Normal |
|---|---|
| Start Angle: | 3 degrees |
| Stop Angle: | 40 degrees |
| Number of Points: | 1851 points |
| Step Size: | 0.02 degrees |
| Datafile Resolution: | 1600 |
| Scan Rate: | 0.04 |
| Scan Mode: | Step |
| Wavelength: | 1.540562 |

Diffraction Optics:

Detector:

Type: Fixed Slits
X2 Configuration: No

Tube:

Type: Fixed Slits
X2 Configuration: No

The results are shown in FIG. 6. At ambient conditions, the XRPD profile of venlafaxine hydrochloride monohydrate exhibited a highly crystalline phase (designated “venlafaxine monohydrate” in FIG. 6).

The sample of venlafaxine hydrochloride monohydrate was then subjected to a nitrogen purge (0% relative humidity) for about 6 hours at room temperature. The XRPD for the sample subjected to the nitrogen purge was different from the original venlafaxine hydrochloride monohydrate sample, indicating the formation of the dehydrated (anhydrous) venlafaxine hydrochloride form. See, for example, the new peak at 4.367° 2θ and small shifts of other major peaks.

Intrinsic Dissolution Rate

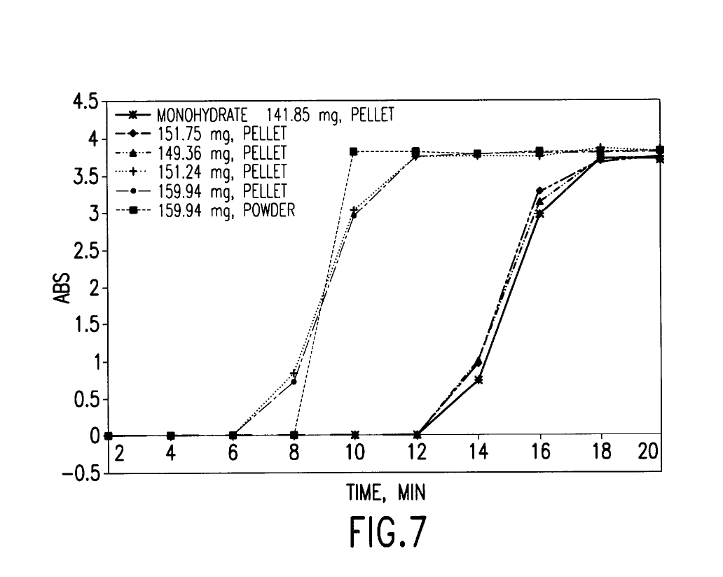

The intrinsic dissolution rates of the raw material venlafaxine hydrochloride and venlafaxine hydrochloride monohydrate were investigated. Pellets of venlafaxine hydrochloride were prepared by compressing 150 mg of each material in a die at 2000 psi for 1 minute with a Carver press. The pellets produced were fitted into stainless steel rings and one side of the ring was coated with paraffin wax which resulted in a single exposed surface of the pellet with a surface area of 1.327 cm². The dissolution rate in 900 mL of water was determined with the USP method (USP 23 (1995), Section 711, Page 1791) in a dissolution apparatus (Vankel 7000) equipped with a Cary 300 Ultraviolet/Visible Spectrophotometer, with a rotation speed of 100 rpm at 37° C. The distance between the lower end of the stirring element and the bottom of the dissolution vessel was set at 2.5 cm. The pellet was centered at the bottom of the flask, and the dissolution media was circulated through a 1.0 cm path microflow cell at a flow rate of ˜10 mL/minute. Absorbance was recorded at 226 nm on a Cary 300 spectrophotometer. The results are shown in FIG. 7.

From the slope of the absorbance versus time profile in FIG. 7, the apparent intrinsic dissolution rates were determined for each sample. Because of the high solubility of venlafaxine hydrochloride in water, only two data points are available for calculation. All of the samples had a dissolution rate of 3.4×10⁻⁴ g/cm²-sec at pH 7.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties. To the extent that a conflict may exist between the specification and a reference, the language of the disclosure made herein controls.

What is claimed is:

1. Venlafaxine hydrochloride monohydrate.

2. Substantially pure venlafaxine hydrochloride monohydrate.

3. Venlafaxine hydrochloride having an X-ray powder diffraction pattern substantially the same as that shown in FIG. 1.

4. Venlafaxine hydrochloride monohydrate exhibiting an X-ray powder diffraction pattern having characteristic peaks expressed in degrees 2θ at about 7.45, 8.60, 12.93, 14.95, 15.54, 15.84, 16.43,17.53, 18.02, 18.58, 18.80, 19.54, 20.05, 20.51, 21.41, 22.46, 23.07, 23.52, 24.08, 24.70, 25.44, 26.54, 27.07, 28.29, 30.60, 31.37, 32.25, 32.74, 34.04, and 34.60.

5. Venlafaxine hydrochloride monohydrate exhibiting an X-ray powder diffraction pattern having characteristic peaks expressed in degrees 2θ at about 7.45, 8.60, 14.95, 18.02, 21.41, 27.07, 32.74, and 34.60.

6. A pharmaceutical composition comprising a therapeutically effective amount of an venlafaxine hydrochloride monohydrate and a pharmaceutically acceptable carrier or diluent.

7. The pharmaceutical composition of claim 5, wherein the venlafaxine hydrochloride monohydrate is substantially pure.

8. The pharmaceutical composition of claim 6, wherein the pharmaceutical composition contains less than 1% by weight of crystalline polymorphs of venlafaxine hydrochloride other than venlafaxine hydrochloride monohydrate, based upon 100% total weight of venlafaxine hydrochloride in the pharmaceutical composition.

9. The pharmaceutical composition of claim 5, wherein the composition comprises a plurality of spheroids comprising the venlafaxine hydrochloride monohydrate.

10. A pharmaceutical composition comprising a therapeutically effective amount of an venlafaxine hydrochloride having an X-ray powder diffraction pattern substantially the same as that shown in FIG. 1 and a pharmaceutically acceptable carrier or diluent.

11. The pharmaceutical composition of claim 9, wherein the venlafaxine hydrochloride is substantially pure.

12. The pharmaceutical composition of claim 10, wherein the pharmaceutical composition contains less than 1% by weight of crystalline polymorphs of venlafaxine hydrochloride other than venlafaxine hydrochloride, based upon 100% total weight of venlafaxine hydrochloride in the pharmaceutical composition.

13. The pharmaceutical composition of claim 9, wherein the composition comprises a plurality of spheroids comprising the venlafaxine hydrochloride.

14. A method of treating depression in a mammal in need thereof comprising administering to the mammal an anti-depressive effective amount of venlafaxine hydrochloride monohydrate.

15. A method of treating depression in a mammal in need thereof comprising administering to the mammal an anti-depressive effective amount of venlafaxine hydrochloride having an X-ray powder diffraction pattern substantially the same as that shown in FIG. 1.

16. A method of treating generalized anxiety disorder in a mammal in need thereof comprising administering to the mammal an effective amount of venlafaxine hydrochloride monohydrate.

17. A method of treating generalized anxiety disorder in a mammal in need thereof comprising administering to the mammal an effective amount of venlafaxine hydrochloride having an X-ray powder diffraction pattern substantially the same as that shown in FIG. 1.

18. A method of treating social anxiety disorder in a mammal in need thereof comprising administering to the mammal an effective amount of venlafaxine hydrochloride monohydrate.

19. A method of treating social anxiety disorder in a mammal in need thereof comprising administering to the mammal an effective amount of venlafaxine hydrochloride having an X-ray powder diffraction pattern substantially the same as that shown in FIG. 1.

20. A method of treating post traumatic stress disorder in a mammal in need thereof comprising administering to the mammal an effective amount of venlafaxine hydrochloride monohydrate.

21. A method of treating post traumatic stress disorder in a mammal in need thereof comprising administering to the mammal an effective amount of venlafaxine hydrochloride monohydrate.

22. A method of treating panic disorder in a mammal in need thereof comprising administering to the mammal an effective amount of venlafaxine hydrochloride monohydrate.

23. A method of treating panic disorder in a mammal in need thereof comprising administering to the mammal an effective amount of venlafaxine hydrochloride having an X-ray powder diffraction pattern substantially the same as that shown in FIG. 1.

24. A method of preventing relapse or recurrence of depression in a mammal in need thereof comprising administering to the mammal an effective amount of venlafaxine hydrochloride monohydrate.

25. A method of preventing relapse or recurrence of depression in a mammal in need thereof comprising administering to the mammal an effective amount of venlafaxine hydrochloride having an X-ray powder diffraction pattern substantially the same as that shown in FIG. 1.

26. A method of preparing venlafaxine hydrochloride monohydrate comprising recrystallizing form I or II of venlafaxine hydrochloride or a mixture thereof in water or a mixture of water and one or more organic solvents.

27. A method of preparing venlafaxine hydrochloride monohydrate comprising wet granulating form I or II of venlafaxine hydrochloride or a mixture thereof.
